(19) United States
(12) Patent Application Publication
(10) Pub. No.: US 2020/0345998 A1
(43) Pub. Date: Nov. 5, 2020

(54) THREADED SWIVEL CONNECTOR FOR AIRWAY TUBE

(71) Applicant: COVIDIEN LP, Mansfield, MA (US)

(72) Inventors: Noel Morgan, Athlone (IE); James P. Curley, Via Athlone (IE); Graham B. Fleming, Athlone (IE); Paul J. Gallogly, Roscommon (IE); Katie M. Spitler Bull, Stafford, VA (US); Sean Morris, Ballymurray (IE)

(21) Appl. No.: 16/864,440

(22) Filed: May 1, 2020

Related U.S. Application Data
(60) Provisional application No. 62/843,029, filed on May 3, 2019.

(51) Int. Cl. A61M 39/10; A61M 16/04

(52) U.S. Cl. CPC A61M 39/1055; A61M 2039/1033; A61M 16/0465

(57) ABSTRACT

A connector for an airway tube for delivery of airway gases to a patient includes a male connector having an interior passage. In an embodiment, the connector includes a spigot having a tapered section at a distal end. The connector may include a locking nut encircling the tapered section and having a plurality of interior threads facing an exterior surface of the tapered section. In an embodiment, the male connector, the spigot, and the locking nut are rotatable independently of each other, and a fluid path extends through the male connector and the spigot for delivery of airway gases through the swivel connector

### CROSS-REFERENCE TO RELATED APPLICATIONS

The present application claims priority to and the benefit of U.S. Provisional Application No. 62/843,029 filed on May 3, 2019, the disclosure of which is incorporated by reference in its entirety for all purposes.

### BACKGROUND

The present disclosure relates generally to medical devices and, more particularly, to a threaded connector for an airway tube. Airway tubes such as endotracheal tubes and tracheostomy tubes are used to provide a passageway for breathing gases into patients who are undergoing medical procedures or who have difficulty breathing. These tubes are inserted with the most distal end of the tube in the patient's trachea, and the proximal end exiting the patient's nose, mouth, or surgical opening (such as a stoma for a tracheostomy tube). The proximal end of the tube is connected to a breathing circuit, which is then connected to an air supply. The remainder of this disclosure relates to improvements in this field.

### SUMMARY

Certain embodiments commensurate in scope with the originally claimed subject matter are summarized below. These embodiments are not intended to limit the scope of the disclosure. Features in one aspect or embodiment may be applied as features in another aspect or embodiment, in any appropriate combination. The present disclosure may encompass a variety of forms that may be similar to or different from the embodiments set forth below.

In one embodiment, a swivel connector for an airway tube for delivery of airway gases to a patient includes a male connector having an interior passage and a spigot. The spigot is coupled to the male connector via a male joint at a spigot proximal end and includes tapered section at a spigot distal end. The swivel connector also includes a locking nut encircling a portion of the tapered section and having a plurality of interior threads facing an exterior surface of the tapered section. The male connector, the spigot, and the locking nut are rotatable independently of each other. A fluid path extends through the male connector and the spigot for delivery of airway gases through the swivel connector to a patient.

In one embodiment, a tracheal tube includes an airway tube having a proximal end and a distal end and a connector coupled to the proximal end of the airway tube. The connector includes a male connector having an interior passage and a spigot extending distally from the male connector, wherein the spigot is at least partially inserted into the proximal end of the airway tube. The male connector also includes a locking nut coupled to the spigot and having a plurality of interior threads gripping an exterior surface of the proximal end such that the proximal end is positioned between the locking nut and the spigot. The male connector, the spigot, and the locking nut are rotatable independently of each other. A fluid path extends through the male connector and the spigot for delivery of airway gases through the swivel connector and into the airway tube.

In an embodiment, a threaded connector for an airway tube for delivery of airway gases to a patient includes a male connector having an interior passage. The threaded connector includes a tapered section extending distally from the male connector. An internal bore is formed within the tapered section, and an internal spigot is disposed within the internal bore to form a fluid path with the internal passage. A distal end of the internal spigot extends distally beyond the tapered section. A plurality of threads disposed on a surface of the internal bore face the internal spigot.

Features in one aspect or embodiment may be applied as features in any other aspect or embodiment, in any appropriate combination. For example, any one of system, laryngoscope, controller, introducer, or method features may be applied as any one or more other of system, laryngoscope, controller, introducer, or method features.

### BRIEF DESCRIPTION OF THE DRAWINGS

Advantages of the disclosed techniques may become apparent upon reading the following detailed description and upon reference to the drawings in which:

FIG. 1 is a diagram of a breathing system for an intubated patient, according to an embodiment of the present disclosure.

FIG. 2 is a perspective view of a swivel connector according to an embodiment of the present disclosure.

FIG. 3 is a cross-sectional view of the swivel connector of FIG. 2.

FIG. 4 is a cross-sectional view of the swivel connector of FIG. 2, with an endotracheal tube attached.

FIG. 5 is a perspective view of a threaded connector according to an embodiment of the present disclosure.

FIG. 6 is a cross-sectional view of the threaded connector of FIG. 5.

FIG. 7 is a detail cross-sectional view of the threaded connector of FIG. 5.

### DETAILED DESCRIPTION OF SPECIFIC EMBODIMENTS

One or more specific embodiments of the present techniques will be described below. According to an embodiment, a connector for an airway tube is provided with one or more swivel joints, enabling relative rotation between the airway tube and breathing circuit. In an embodiment, the connector includes threads for threaded attachment to the airway tube, to reduce the incident of unintended disconnection, as described further below. The connector for the airway tube may be used in conjunction with airway tubes that are inserted into a patient and that are coupled to components of the breathing circuit.

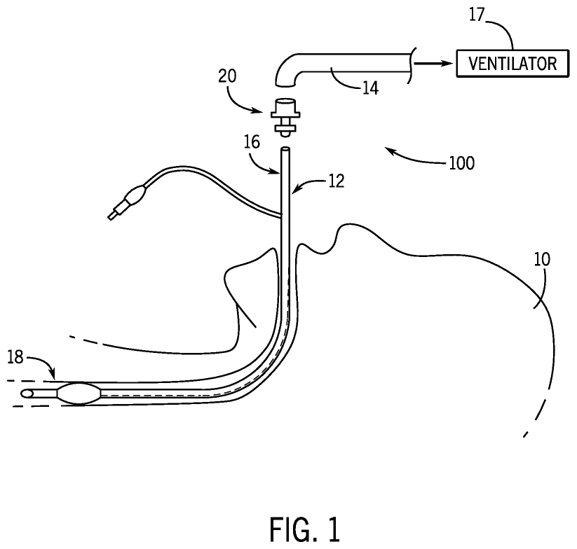

FIG. 1 shows a breathing system 100 according to an embodiment of the invention. The system includes a patient 10 intubated with an airway tube 12, in this case an endotracheal tube. A threaded swivel connector 20 connects the airway tube 12 to a breathing circuit 14, which in turn connects to an air supply 17, such as a ventilator. Inhalation breathing gases for the patient flow from the air supply 17, through the tubular breathing circuit 14, through an internal passage in the connector 20, into the airway tube 12, and into the patient's lungs (and vice versa, for exhalation).

In this situation, it is important that the components along the airflow path remain connected, so that the airflow path stays intact and can adequately deliver breathing gases to the patient. According to embodiments herein, the threaded swivel connector 20 provides a reliable connection between the airway tube 12 and the breathing circuit 14, reducing the chance of inadvertent disconnection. The threads on the connector provide a connection to the airway tube that is both secure and removable. The connector 20 can be manually unscrewed by an operator to remove the connector 20 from the airway tube 12 if needed. Though inadvertent disconnection of the connector 20 from the tube 12 is problematic, there are several reasons that a healthcare worker may want to intentionally disconnect the connector 20 from the tube 12.

For example, after the airway tube 12 is placed in the patient, the healthcare worker may want to cut the tube 12 to a shorter size. Referring to FIG. 1, the airway tube 12 includes a proximal end 16 exiting the patient's airway and an opposite distal end 18 inserted into the patient's trachea. After intubation, a clinical caregiver may want to cut the proximal end 16 to shorten it, so that less length of tube is extending out of the patient's mouth or nose. Shortening the airway tube 12 and the length of the airway tube 12 extending from the patient's mouth or nose can reduce the chance of kinks or twisting of the tube, or catching the tube on other components or accessories near the patient. A shorter proximal end 16 outside of the patient's mouth or nose can be easier to manage and can result in less twisting or torqueing of the tube as other components are connected. A shorter tube 12 also has less dead space, in which air is recirculated without participating in gas exchange in the patient's lungs.

As another example, a clinical caregiver may remove the connector 20 in order to attach a suction line (such as closed suctioning) to the airway tube 12, to attach different air supply such as a high flow therapy or jet ventilation, to temporarily insert a working device into the airway, or to exchange a different sized connector for a better fit. As described in more detail below, the threads on the connector 20 enable the connector to be removed (e.g., manually removed) from the airway tube 12 more easily than a force fit or friction fit connection. With a force fit or friction fit connection between a connector and airway tube, a caregiver may have to twist, torque, and pull on the components to separate them, which can damage the airway tube 12 (such as causing kinks) and can cause discomfort to the patient. If the patient has already been intubated, manipulation of the airway tube 12 to remove a friction fit connector may result in the airway tube 12 rubbing against the patient's tracheal wall or vocal cords, which can cause discomfort. By contrast, the threaded connector 20 can be removed more easily by unscrewing it from the tube 12, so the tube 12 retains its shape.

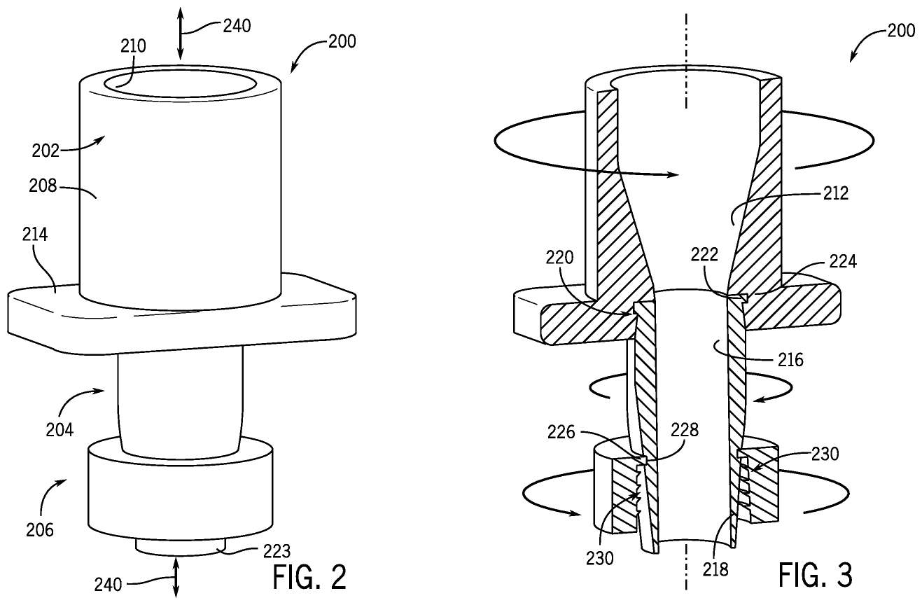

FIGS. 2 and 3 show a threaded swivel connector 200 according to an embodiment of the disclosure. FIG. 2 shows a perspective side view, and FIG. 3 shows a cross-sectional view. In the embodiment shown, the connector 200 includes three components: a male connector 202, a spigot 204, and a twist-lock nut 206. The male connector 202 has a generally cylindrical outer profile 208, and a proximal opening 210 leading into an interior passage 212 that tapers into a funnel. The male connector 202 also has an exterior shelf 214 at a distal end of the connector 202. The male connector 202 is shaped to fit into the breathing circuit 14 (see FIG. 1). The shelf 214 acts as a finger grip to ease connection and disconnection of the breathing circuit 14 when the male connector 202 is fully inserted into the breathing circuit 14.

The spigot 204 provides an interior passage 216 for air flow and connects to the male connector 202 at a swivel joint 220. The spigot 204 has a male snap fit connection at its proximal end and narrows into a tapered section or nozzle 218 at its distal end 223 such that an outer diameter of the spigot 204 is smaller at the distal end 223 relative to a more proximal portion of the spigot 204. The snap fit connection includes a prong 222 that snap fits into a corresponding indentation or recess 224 in the male connector 202, forming the swivel joint 220.

The twist-lock nut 206 encircles the tapered section 218 of the spigot 204. The nut 206 includes a prong 226 that fits into a corresponding indentation or recess 228 in the outer surface of the tapered section 218, so that the nut 206 is carried by the spigot 204. The nut can rotate freely within the indentation or recess 228, which may be formed as an annular groove 360-degrees around the outer surface of the spigot 204. In an embodiment, the spigot 204 has a ribbed or threaded exterior surface that mates with the nut 206. A gap is formed between the nut 206 and the spigot 204 that receives the airway tube 12.

The nut 206 includes internal threads 230 on an inside surface of the nut, facing the spigot 204. The internal threads 230 grip the exterior surface of the airway tube 12 to securely connect the airway tube 12 to the connector 200. By rotating the nut 206 in a first direction, the airway tube 12 is drawn up along the threads between the nut 206 and spigot 204, until the airway tube 12 is pinched firmly between them. To release the airway tube 12 from the connector, the nut 206 is rotated the opposite direction, to push the airway tube 12 back down the threads 230 until it exits the nut 206. Accordingly, the nut 206 may be rotated in a clockwise and counterclockwise direction. In an embodiment, the number of threads provided in the nut is four, though more or fewer could be used. The threads may be right handed or left handed threads, single or multi start threads, or other variations.

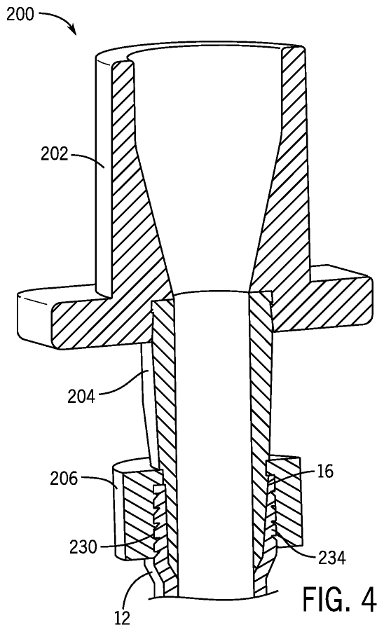

FIG. 4 shows the swivel connector 200 with an endotracheal tube attached. At the bottom of the figure, the proximal end of an endotracheal tube extends up over the tapered section of the spigot and is engaged by the threads between the nut and spigot.

The swivel connector 200 creates a fluid path 240 (FIG. 2) through the connector 200 for passage of breathing gases from the breathing circuit 14 (FIG. 1) to the airway tube 12 via coupling of a connector or tubing of the breathing circuit 14 at the male connector 202. In an embodiment, the male connector 202 is configured as a 15mm connector, e.g., a 15mm ISO connector with geometry configured according to the ISO standard. The connector 200 can be securely connected to the breathing circuit 14 via the twist-lock nut 206, which grips the tube 12 with the internal threads 230 and securely seals the tube 12 against the outer surface of the tapered section 218 of the spigot 204. The swivel connector 200 can also be conveniently removed from the tube 12 by twisting the lock 206 to unthread and remove the airway tube 12.

As shown in FIG. 2, the connector 200 includes three components that rotate with respect to each other about the same axis of rotation. The nut 206 rotates around the spigot 204 to engage an airway tube 12 to the threads 230, as described above. Once the airway tube 12 is sealed between the nut 230 and the spigot 204, the nut and spigot are locked together, and no longer freely rotate relative to each other. The swivel joint 220 enables relative rotation between the male connector 202 and the spigot 204. When the spigot 204 is sealed against the tube 12 and the male connector 202 is inserted into the breathing circuit 14, the swivel joint 220 introduces relative rotation between the airway tube 12 and the breathing circuit 14. This relative rotation insulates the airway tube 12 from rotational forces exerted on the male connector 202 and circuit 14, such as twisting, repositioning, and other movements. This relative rotation is accomplished without adding a separate component outside of or around the male connector 202. The male connector 202 can be directly attached to the breathing circuit 14 while still providing relative rotation between the breathing circuit 14 and the airway tube 12.

The joint 220 can be created with any suitable rotatable connection, such as a snap-fit (shown FIG. 2), a bearing insert, an O-ring or gasket.

In an embodiment, the components of the connector 200 are all made of the same material. In an embodiment, the material is rigid, to provide good compressive force against the airway tube and to maintain the inner diameter for passage of the breathing gases.

In an embodiment, a set of connectors 200 is provided, including several connectors 200 with differently sized spigots 204 and nuts 206 for connection to differently sized airway tubes. For example, endotracheal tubes come in a variety of sizes (e.g., having different internal diameters), to fit into tracheas of different sizes (such as smaller neonatal or pediatric tubes for babies or children, and larger tubes for adults). The spigot 204 of the connector 200 is sized to fit inside the proximal open end of the endotracheal tube. Accordingly, a set of connectors 200 includes different connectors each with smaller or larger spigots 204 (and corresponding encircling nuts 206) that are sized to accommodate smaller or larger airway tubes. In an embodiment, a different connector 200 is provided for each different size of airway tube, to make sure the connector makes a good seal with the tube.

The connector 200 can be used with various types of airway tubes, such as endotracheal tubes, oral or nasal tubes, tracheostomy tubes, nasal prongs, or other airway interfaces.

In an embodiment, the swivel joint 220 is omitted and the male connector 202 and spigot 204 are fixedly attached together, or made of one continuous piece. In this embodiment, the threaded nut 206 still rotates with respect to the spigot, to retain an airway tube. However, once the airway tube is sealed between the nut and spigot, the connector will not permit relative rotation between the circuit 14 and airway tube 12.

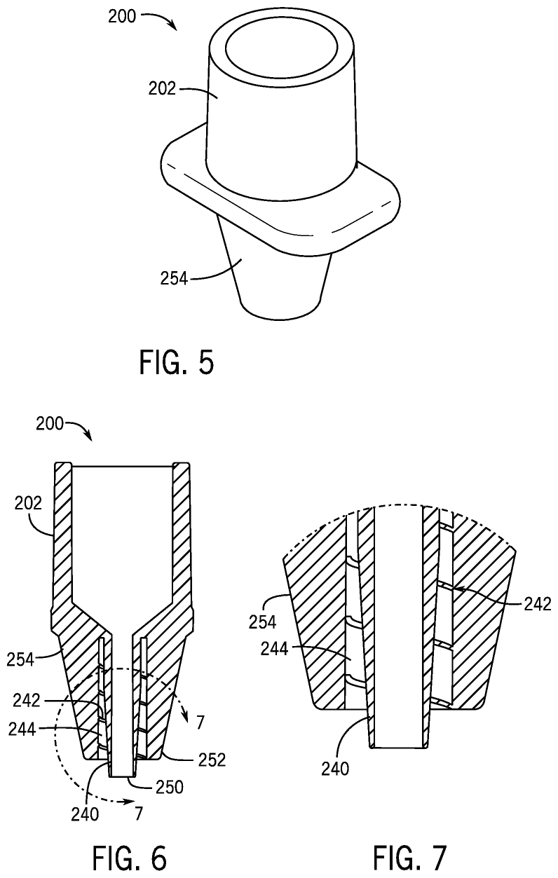

FIG. 5 shows a perspective view and FIG. 6 shows cross-sectional views of a threaded connector according to an embodiment of the disclosure. FIG. 7 is a detail view of FIG. 6. In this embodiment, the connector 200 does not swivel or rotate, but provides an interior spigot 240 for insertion into an airway tube, and interior threads 242 for gripping the exterior surface of the airway tube. The interior threads 242 are formed on an internal bore 244 that faces the interior spigot 240. The interior spigot 240 is spaced apart from and forms a gap with the internal bore 244. The gap may narrow towards its proximal end (e.g., terminating distally of the male connector 202 or a funnel of the interior passage of the male connector 202). The interior spigot 240 may protrude such that a spigot distal end 250 is more distal than a distal end 252 of a tapered section 254. This design provides a connector that securely seals against an airway tube and that can be conveniently connected or disconnected by threading or unthreading the airway tube from the connector.

As disclosed herein, a connector with threads is able to securely grip an airway tube to reduce the likelihood that the airway tube is unintentionally or inadvertently disconnected from the connector. Connectors without threads, such as force-fit connectors, are inserted into an airway tube and rely on the friction between the connector and tube to prevent disconnection. The amount of force needed to remove this connector may depend on the depth of insertion of the connector into the tube, which can vary with clinical practice. In the event a user does not insert the force-fit connector fully into the airway tube, the force-fit connector may be too easily disconnected. In an embodiment, the disconnect force that is needed to remove a threaded connector according to embodiments herein from an airway tube is higher than the disconnect force to remove a non-threaded connector (such as force-fit or friction-fit). Additionally, the disconnect force for a threaded connector according to embodiments herein is more consistent than the disconnect force for a non-threaded connector, because by twisting the threaded connector through the number of turns to seat the airway tube against the threads, the connection is more consistently made to a sufficient depth.

In an embodiment, the threaded connector or threaded swivel connector described herein is connected to an airway tube that is not flared at its proximal end. That is, the airway tube 12 (see FIG. 1) retains a uniform or consistent inner and outer diameter at its proximal end 16. The threads on the connector facilitate insertion of the connector into the proximal end 16 of the airway tube 12 without the need for a flared or wide proximal end to guide the connector into the tube.

In an embodiment, the connector is manufactured via injection molding and, in an embodiment, the threaded swivel connector is assembled via snap-fit assembly. In an embodiment, the threaded connector may be a unitary or integrally formed assembly.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A swivel connector for an airway tube for delivery of airway gases to a patient, comprising:
a male connector, the male connector comprising an interior passage;
a spigot coupled to the male connector via a male joint at a spigot proximal end and comprising a tapered section at a spigot distal end; and
a locking nut encircling a portion of the tapered section and comprising a plurality of interior threads facing an exterior surface of the tapered section,
wherein the male connector, the spigot, and the locking nut are rotatable independently of each other, and
wherein a fluid path extends through the male connector and the spigot for delivery of airway gases through the swivel connector.

2. The swivel connector of claim 1, wherein the tapered section comprises a groove, and wherein the locking nut is rotatable within the groove.

3. The swivel connector of claim 2, wherein the locking nut comprises a prong that fits into the groove.

4. The swivel connector of claim 1, wherein the male connector is a 15mm connector.

5. The swivel connector of claim 1, wherein the tapered section forms a nozzle at the spigot distal end.

6. The swivel connector of claim 1, wherein the tapered section comprises a ribbed or threaded exterior surface that faces the locking nut.

7. The swivel connector of claim 1, wherein the male connector comprises an interior recess at a distal end of the male connector, wherein the interior recess mates with the male joint of the spigot to form a swivel joint.

8. The swivel connector of claim 1, comprising a shelf extending laterally from a distal end of the male connector.

9. The swivel connector of claim 1, wherein a gap is formed between the locking nut and the tapered section, wherein the gap is configured to receive the airway tube.

10. A tracheal tube, comprising:
an airway tube comprising a proximal end and a distal end;
a connector coupled to the proximal end of the airway tube, wherein the connector comprises:
a male connector comprising an interior passage;
a spigot extending distally from the male connector, wherein the spigot is at least partially inserted into the proximal end of the airway tube; and
a locking nut coupled to the spigot and comprising a plurality of interior threads gripping an exterior surface of the proximal end such that the proximal end is positioned between the locking nut and the spigot,
wherein the male connector, the spigot, and the locking nut are rotatable independently of each other, and
wherein a fluid path extends through the male connector and the spigot for delivery of airway gases through the swivel connector and into the airway tube.

11. The tracheal tube of claim 10, wherein the male connector is a 15mm connector.

12. The tracheal tube of claim 10, wherein the male connector is coupled to a breathing circuit of a ventilator.

13. The tracheal tube of claim 10, wherein the spigot comprises a groove and wherein the locking nut is coupled to the spigot via a prong that fits into the groove.

14. The tracheal tube of claim 10, wherein the locking nut is rotatable in a clockwise direction and a counterclockwise direction.

15. The tracheal tube of claim 10, wherein the interior passage of the male connector forms a funnel that narrows towards a passage of the fluid path of spigot.

16. A threaded connector for an airway tube for delivery of airway gases to a patient, comprising:
a male connector, the male connector comprising an interior passage;
a tapered section extending distally from the male connector;
an internal bore formed within the tapered section;
an internal spigot disposed within the internal bore and forming a fluid path with the internal passage, wherein a distal end of the internal spigot extends distally beyond the tapered section; and
a plurality of threads disposed on a surface of the internal bore facing the internal spigot.

17. The threaded connector of claim 16, wherein the male connector and the tapered section are fixedly coupled to one another or formed as a unitary assembly.

18. The threaded connector of claim 16, wherein the internal spigot and the surface of the internal bore are spaced apart from one another to form a gap that is configured to receive the airway tube.

19. The threaded connector of claim 18, wherein a proximal end of the gap terminates distally of a funneled section of the interior passage.

20. The threaded connector of claim 18, wherein the gap narrows towards the male connector.
